(19) United States
(12) Patent Application Publication
(10) Pub. No.: US 2015/0374817 A1
(43) Pub. Date: Dec. 31, 2015

(54) PHARMACEUTICAL COMPOSITION FOR THE TREATMENT OF BURNOUT SYNDROME

(71) Applicants: Anja BILL, Würzburg (DE); Josip S. BILL, Würzburg (DE)

(72) Inventor: Josip S. BILL, Würzburg (DE)

(21) Appl. No.: 14/769,716

(22) PCT Filed: Feb. 21, 2014
(86) PCT No.: PCT/EP2014/053424

§ 371 Date: Aug. 21, 2015

(30) Foreign Application Priority Data
Feb. 22, 2013 (EP) 13156318.1

(51) Int. Cl. A61K 39/395; A61K 31/355; A61K 31/375; A61K 31/59; A61K 31/51; A61K 33/30; A61K 31/714; A61K 31/4415; A61K 31/525; A61K 31/4188; A61K 33/26; A61K 33/06; A61K 31/07; A61K 31/593

(52) U.S. Cl. CPC A61K 39/39516; A61K 31/07; A61K 31/355; A61K 31/375; A61K 31/59; A61K 31/51; A61K 31/593; A61K 31/714; A61K 31/4415; A61K 31/525; A61K 31/4188; A61K 33/26; A61K 33/06; A61K 33/30

(57) ABSTRACT

The present invention is directed to a pharmaceutical composition or a kit-of-parts and its use in the treatment of burnout syndrome and longevity therapy.

The present invention is directed to a pharmaceutical composition or a kit-of-parts and its use in the treatment of burnout syndrome and longevity therapy.

### BACKGROUND OF THE INVENTION

Burnout is the psychological term for the experience of long-term exhaustion and diminished interest. Burnout is classified in Z73 of ICD-10 named “life-management difficulties”. The European Agency for Safety and Health at Work estimates that the economic losses due to the burnout syndrome in the EU amount to approximately 22 billion Euros per year. Although there are attempts to prevent the burnout syndrome by appropriate measures focusing on work load, reward, control, community, fairness and values, the burnout syndrome seems to be relevant for an increasing number of working people.

Although no final figures are available it seems that burnout has become already a widespread disease. By results of a representative survey in 2011, physicians have diagnosed a burnout syndrome for 1.9 million people aged 14 to 65 years in Germany. Further, in the light of healthcare politics, 1.8 million sick leaves in 2010 due to a burnout are economically a huge burden.

Therapies, which are presently used for the treatment of a burnout are, among others, psychotherapy, especially cognitive behavioral therapy (CBT), phytotherapy, physiotherapy, adjuvant pharmacotherapy and complementary treatments like music therapy or body-mind therapies. The therapy of burnout depends mainly on the understanding of burnout, whether it is regarded as independent disease, as a preliminary stage of a depression or as a co-morbidity of depression.

US 2003/0206898 A1 discloses the use of a vitamin mixture in combination with antibody D2E7 for the treatment of rheumatoid arthritis. However, this document does not refer to a precise vitamin mixture but merely suggests the use of “multivitamins”.

WO 2009/023411 describes a composition containing an antibody (anti-human OPN antibody) and vitamins C and E and salts thereof. However, the use of IgG antibodies is not disclosed in this document. Further, the document does not disclose the use of an IgG antibody/vitamin mixture for the treatment of burnout syndrome and for longevity therapy.

WO 00/64439 is directed to the use of a composition comprising an antioxidant (for example vitamins A, C or E) for ameliorating the side effects of radio immune therapy. This document, however, is not related to the therapy of the burnout syndrome or to the longevity therapy. Further, this document does not disclose the use of IgG antibodies.

The paper “Neonatal Hemochromatosis: a case report”, Lauree Pearson et al, Official Journal of the National Association of Neonatal Nurses, April 2009, describes an anti-oxidative therapy by using vitamins C and E and immunoglobulins.

The efficacy of therapies for the treatment of the burnout syndrome still is insufficiently investigated. Therefore, there is still a demand for medical therapies which address the physical and psychological needs of people suffering from burnout syndrome. Further, there is still a need for new approaches in longevity therapy.

### DEFINITIONS

The term “composition” as used herein is intended to mean a composition for use in the administration to the human or animal body. Therefore, the composition according to the invention can be defined as a pharmaceutical or a therapeutic composition as well. Such a composition contains the active ingredients along with non-active components or excipients. The composition according to the invention can take the form of several types of dosage forms, including liquid dosage forms, solid dosage forms and semi-solid dosage forms which can also be differentiated by the method of administration.

A “kit-of-parts” according to the present invention means a composition of the active ingredients, further containing non-active ingredients, where the different components are not present in mixed, but spatially distinct form. An example of a kit-of-parts would be an assembly containing an injection solution comprising IgG antibodies and at least one component comprising a vitamin mixture which is, for example, in tablet or liquid form for oral administration.

According to standard definition, a composition comprises the different components in intimately mixed form, whereas a kit-of-parts contains the component in spatially distinct form.

### BRIEF DESCRIPTION OF THE INVENTION

A first aspect of the present invention relates to a composition or kit-of-parts comprising or consisting of

a) at least one component comprising a vitamin mixture; and

b) a component comprising IgG antibodies.

A second aspect of the present invention is related to such a composition or kit-of-parts for use in the treatment of burnout syndrome or in longevity therapy.

### DETAILED DESCRIPTION OF THE INVENTION

According to the first aspect of the invention, a composition or kit-of-parts comprises or consists of

a) at least one component comprising a vitamin mixture; and

b) a component comprising IgG antibodies.

The composition and the kit-of-parts will take the form of a pharmaceutical or therapeutic composition, i.e. will contain or consist of one or more active ingredients and one or more non-active ingredients or excipients.

According to the present invention, different options are available regarding the dosage form in which the composition or kit-of-parts according to the invention is present.

Component a) of the present composition or kit-of-parts, i.e. the at least one component comprising a vitamin mixture, might take different forms suitable for different ways of administration. Thus, the vitamin mixture may be present in a form suitable for oral administration, i.e. in form of soft or hard shell capsules. Soft and hard shell capsules are widely used within the pharmaceutical and health food industry and have gained broad acceptance as they present pharmaceutical products in a form that is readily to be consumed and digested by a user. These capsules generally are made up of a shell and an active filling material. The shell is formed of readily digested materials, for example, a soft gelatin capsule which might comprise a mixture of gelatin, glycerol and water. Hard shell capsules generally comprise gelatin and water.

Water soluble vitamins such as B-group vitamins and ascorbic acid (vitamin C) are generally present in the form of a suspension in edible oil when encapsulated in a soft gelatin or hard shell capsules. For example, oils such as soya bean oil are generally used. Therefore, according to the present invention, component a) may be present in form of a soft or hard shell capsule comprising the mixture of the preferred vitamin ingredients.

Should soft shell capsules with fill-in liquids be used, the invention may be based on any liquid base system that can be encapsulated in a soft (or even hard) shell capsule. Examples of hydrophobic fill-in liquids include vegetable oil, vegetable oil derivatives or medium-chain triglycerides or mixtures thereof. Preferred examples of those vegetable oils include almond oil, arachis oil, borage oil, canola oil, fractionated coconut oil, lecithin, maize oil, olive oil, rapeseed oil, rice bran oil, soya bean oil, sunflower oil and many others.

Hydrophilic fill-in liquids include PEG's of different molecular weight (preferably from 300 to 8,000).

The soft or hard shell capsule may include any suitable amount of vitamin particles in suspension, but generally will include from 10 mg to 1,000 mg of vitamin as an active ingredient. The vitamin composition may include a mixture of coated vitamin particles or a combination of coated particles and other coated or uncoated active ingredients.

As an alternative, the vitamin component may take the form of a tablet or of a swallowable liquid such as a syrup or drops. In the latter case, the liquid might contain fruit flavours, odors and other additives to improve smell and taste of the liquid composition.

As a further alternative, component a) might take the dosage form of a parenteral composition, such as a solution for intravenous infusion or injection. Thus, the component advantageously may be provided as a formulation for infusion. This may be a pre-concentrate, to be diluted prior to administration, or a ready-to-use solution, fat emulsion or a dispersion. Regarding the scientific and legal requirements of manufacturing and administering parenteral solutions it is referred to Remington, The Science and Practice of Pharmacy, 22^{nd} Edition, 2012. In particular, chapter 41, the formulation principles, vehicles and solutes as well as manufacturing processes gives a clear guidance on the manufacture of parenteral compositions.

The vitamins may be selected from the group of water soluble vitamins such as B- and C-group vitamins mentioned above or fat soluble vitamins such as vitamin H, vitamin A, vitamin D or vitamin E.

In a preferred embodiment, the vitamin mixture of the present invention comprises ascorbic acid, biotin, thiamine pyrophosphate, colecalciferol, cyanocobalamin, panthenol, folic acid, nicotinamide, pyridoxine hydrochloride, retinol palmitate, riboflavin-5′-phosphate and/or α-tocopherol.

In a particularly preferred embodiment, the following vitamin mixture according to table 1 is used as part of an infusion solution of 500 ml.

TABLE 1

| | |
|---|---|
| Ascorbic acid | 100-150 mg, preferably 125 mg |
| Biotin | 50-100 μg, preferably 69.00 μg |
| Thiamin | 2-4 mg, preferably 3 mg |
| Colecalciferol | 3-7 μg, preferably 5.0 μg |
| Cyanocobalamin | 4-8 μg, preferably 6.0 μg |
| Dexpanthenol | 14-18 mg, preferably 16 mg |
| Folic acid | 410-420 μg, preferably 414.00 μg |
| Nicotinamide | 40-50 mg, preferably 46.00 mg |
| Pyridoxine hydrochloride | 3-7 mg, preferably 5 mg |
| Retinol palmitate | 0.5-1.5 mg, preferably 1.0 mg |
| Riboflavin | 3-5 mg, preferably 4.0 mg |
| α-Tocopherol | 5-15 mg, preferably 10 mg |

Additionally, component a) may comprise minerals such as trace elements of iron, calcium, magnesium and zinc etc. in order to provide an additional benefit for the patient.

The mineral additives to be added to the vitamin mixture might be as follows (calculated based on an infusion of 500 ml):

TABLE 2

| | |
|---|---|
| K⁺ | 15-25, preferably 20.00 mmol |
| Mg²⁺ | 4-6, preferably 5.00 mmol |
| Na⁺ | 15-25, preferably 20.00 mmol |
| Zn²⁺ | 10-14, preferably 12.00 μmol |
| Cu²⁺ | 4-8, preferably 6.00 μmol |
| Co²⁺ | 2-6, preferably 4.00 μmol |
| Mn²⁺ | 2-6, preferably 4.00 μmol |

It is noted that the above indicated preferred concentration ranges (see tables 1 and 2) may vary widely, for example in a range of ±50%, preferably ±20% of the preferred concentration.

The second component of the composition or kit-of-part according to the present invention comprises IgG antibodies. Immunoglobulin G (IgG) is an antibody isotype. It is a protein complex composed of four peptide chains, namely two identical heavy chains and two identical light chains. There are four IgG subclasses in humans named in order of their abundance in serum (IgG1 being the most abundant): IgG1, IgG2, IgG3 and IgG4. Therefore, the IgG antibodies according to element b) of the present composition or kit-of-part usually will be a mixture of differing amounts of IgG1 to IgG4.

Preferably, the ratio between IgG1, 2, 3 and 4 used in component b) reflects the natural abundance of the IgG subclasses in serum. That is to say, IgG1 contributes to about 66%, IgG2 to about 23%, IgG3 to about 7% and IgG4 to about 4% to the overall amount of IgG antibodies. However, a certain degree of variation for each of the IgG subclasses is acceptable and equally effective. For example, the IgG composition might comprise 57% IgG1, 37% IgG2, 3% IgG3, 3% IgG4. Preferably, the IgG antibodies are human blood plasma IgG antibodies.

However, it is also contemplated herein to use other types of antibodies such as monoclonal IgG antibodies. Monoclonal antibodies as used in the present invention are monospecific antibodies that are made by identical immune cells that are all clones of a unique parent cell, in contrast to polyclonal antibodies which are made from several different immune cells. Monoclonal antibodies have monovalent affinity, in that they bind to the same epitope. Monoclonal antibodies may be produced by the well known hybridoma technology developed by Koehler and Milstein.

In a preferred embodiment, the kit-of-parts according to the present invention comprises or consists of at least two components, i.e. at least one component comprising a vitamin mixture and one component comprising IgG antibodies. As outlined above, the kit-of-parts in the present application means that the respective components are not present in mixed but in spatially distinct form. This allows a time-staggered application of the different components as will be outlined below.

The composition or kit-of-parts according to the present invention might be present in a suitable form for parenteral application, preferably in the form of a solution or suspension for injection or infusion.

In a second aspect, the composition or kit-of-parts of the present invention is claimed for use in the treatment of burnout syndrome or in longevity therapy. According to the data contained in the examples, the patients tested showed a quick response to the administered components already after the first day of treatment. Up to now, no side effects (or negative effects at all) have been shown.

In a preferred embodiment, the kit-of-parts of the present invention is used in the treatment of burnout syndrome or in longevity therapy, wherein the treatment/therapy comprises the following steps:

a) administering a component comprising a vitamin mixture;

b) administering a component comprising IgG antibodies; and

c) administering a further component comprising a vitamin mixture to a patient in need thereof.

As an ideal setting for the present treatment, it turned out that steps a) to c) should be performed by intravenous infusion and should have a duration of approximately two hours each. Further, steps a) to c) preferably are repeated five times on five consecutive days followed by a break of one to two days, which in turn is followed by a five times repetition of steps a) to c) on five consecutive days.

Therefore, one single “treatment cycle” takes about five days which is interrupted by a break, where, optionally, a further treatment cycle is added after the break.

In a further preferred embodiment, the composition or kit-of-parts of the present invention is used in the treatment of burnout syndrome or the longevity therapy, where the patients treated belong to the elderly population.

In the following, the present invention is further illustrated by examples. They should in no case be interpreted as a limitation of the scope of the invention as defined in the claims.

### EXAMPLES

A group of patients according to Table 3 was subjected to the following anti-burnout and longevity treatment schedule:

Day 1 to 5:
vitamin intravenous infusion of 500 ml over 2 hours
antibody intravenous infusion (human blood plasma IgG (57% IgG1, 37% IgG2, 3% IgG3, 3% IgG4)), 200 ml over 2 hours
vitamin intravenous infusion of 500 ml over 2 hours
   on each day.

The vitamin intravenous infusion was as defined in Table 1.

Following the first treatment cycle, a break of one to two days occurred followed by a second therapy cycle on therapy days six to ten:
vitamin intravenous infusion of 500 ml over 1 hour
antibody intravenous infusion (human blood plasma IgG (57% IgG1, 37% IgG2, 3% IgG3, 3% IgG4)), 200 ml over 1 hour
vitamin intravenous infusion of 500 lm over 1 hour

The aim of the therapy was an IgG plasma level of a minimum of 4 to 6 g/l.

In Table 3, the results of an overall number of 20 patients along with their personal data (gender, age, pre-existing diseases, diagnosis etc.) are summarized.

TABLE 3
Patients treated according to the present invention

| | | | | | | | | | | | Additional | Improvement |
|---|---|---|---|---|---|---|---|---|---|---|---|---|
| | | | | | | | | | | | diseases | of preexisting |
| | | | | | Pre- | | | | | | occurring | diseases |
| | | | | Date of | exist. | | | VAS prior to | VAS after | VAS 12 | within 24 | within 24 |
| No. | Abbr. | Gender | Age | treatment | Disease | Diagnosis | Therapy | therapy | therapy | months later | months | months |
| 01 | G.B. | M | 55 | 7.-11.2.05 | HYP | BurnOut | Comb. th. | 2 | 10 | 9 | No | Yes |
| 02 | F.T. | M | 59 | 14.-18.2.05 | oB | BurnOut | Comb. th. | 2 | 10 | 10 | No | ./. |
| 03 | E.R. | M | 52 | 14.-18.2.05 | oB | BurnOut | Comb. th. | 2 | 10 | 10 | No | ./. |
| 04 | L.L. | M | 55 | 19.-23.2.05 | oB | BurnOut | Comb. th. | 1 | 10 | 10 | No | ./. |
| 05 | H.M. | M | 61 | 4.-8.4.05 | oB | BurnOut | Comb. th. | 1 | 10 | 10 | No | ./. |
| 06 | T.B. | M | 50 | 13.-17.6.05 | DM | BurnOut | Comb. th. | 2 | 10 | 10 | No | No |
| 07 | R.K. | M | 62 | 5.-9.9.05 | oB | BurnOut | Comb. th. | 2 | 10 | 9 | No | ./, |
| 08 | A.H. | M | 63 | 23.-27.1.06 | oB | BurnOut | Comb. th. | 1 | 10 | 10 | No | ./. |
| 09 | G.W. | M | 55 | 13.-17.3.06 | oB | BurnOut | Comb. th. | 2 | 10 | 10 | No | ./. |
| 10 | L.H. | M | 64 | 3.-7.7.06 | KHK | BurnOut | Comb. th. | 3 | 10 | 10 | No | No |
| 11 | A.G. | M | 60 | 10.-14.7.06 | oB | BurnOut | Comb. th. | 2 | 10 | 10 | No | ./. |
| 12 | T.Z. | M | 53 | 2.-6.10.06 | oB | BurnOut | Comb. th. | 1 | 10 | 9 | No | ./, |
| 13 | B.H. | F | 59 | 27.11.- | oB | BurnOut | Comb. th. | 1 | 10 | 10 | No | ./. |
| | | | | 1.12.06 | | | | | | | | |
| 14 | J.M. | M | 54 | 11.-15.12.06 | oB | BurnOut | Comb. th. | 1 | 10 | 10 | No | ./. |
| 15 | E.H. | M | 71 | 19.-23.2.07 | KHK | BurnOut | Comb. th. | 1 | 10 | 9 | No | No |
| 16 | J.G. | F | 59 | 26.2.-2.3.07 | HYP | BurnOut | Comb. th. | 1 | 10 | 10 | No | Yes |
| 17 | M.S. | M | 51 | 5.-9.3.07 | oB | BurnOut | Comb. th. | 2 | 10 | 10 | No | ./. |
| 18 | A.K. | M | 42 | 7.-11.5.07 | oB | BurnOut | Comb. th. | 1 | 10 | 10 | No | ./. |
| 19 | L.B. | M | 54 | 22.-26.10.07 | oB | BurnOut | Comb. th. | 1 | 10 | 10 | No | ./. |
| 20 | J.R. | M | 66 | 29.10.- | oB | BurnOut | Comb. th. | 1 | 10 | 10 | No | ./. |
| | | | | 2.11.07 | | | | | | | | |

Legend:
oB = without medical finding
HYP = hypertension
KHK = coronary heart disease
DM = diabetes mellitus
Therapy:
comb. th. = combination therapy (see above)
VAS = visual analogue scale according to Hayes and Patterson (1921), scale 0-10:
0 points: worst impression of the general physical and mental performance
10 points: best individual impression of the general physical and mental performance

TABLE 4
Control group (treated by psychotherapy)

| | | | | | | | | | | | Additional | Improvement |
|---|---|---|---|---|---|---|---|---|---|---|---|---|
| | | | | | | | | | | | diseases | of preexisting |
| | | | | | Pre- | | | | | | occurring | diseases |
| | | | | Date of | exist. | | | VAS prior to | VAS after | VAS 12 | within | within 24 |
| No. | Abbr. | Gender | Age | treatment | Disease | Diagnosis | Therapy | therapy | therapy | months later | 24 months | months |
| 01 | P.T. | M | 69 | 2.4.-2.5.03 | oB | BurnOut | Iso/PG/P | 2 | 6 | 4 | No | No |
| 02 | A.G. | M | 72 | 4.-25.4.03 | KHK | BurnOut | Iso/PG/P | 2 | 9 | 9 | No | No |
| 03 | K.M. | M | 54 | 1.-29.6.03 | HYP | BurnOut | Iso/PG/P | 3 | 6 | 3 | No | No |
| 04 | J.M. | F | 69 | 26.7- | KHK, | BurnOut | Iso/PG/P | 2 | 5 | 4 | No | No |
| | | | | 14.8.03 | DM | | | | | | | |
| 05 | B.B. | F | 51 | 30.9.- | oB | BurnOut | Iso/PG/P | 1 | 4 | 1 | No | No |
| | | | | 30.10.03 | | | | | | | | |
| 06 | E.S. | M | 49 | 5.-29.10.03 | oB | BurnOut | Iso/PG/P | 3 | 6 | 2 | No | No |
| 07 | D.L. | M | 69 | 7.-28.9.03 | oB | BurnOut | Iso/PG/P | 2 | 7 | 2 | No | No |
| 08 | J.R. | F | 73 | 20.10.- | HYP, | BurnOut | Iso/PG/P | 1 | 7 | 2 | No | No |
| | | | | 22.11.03 | DM | | | | | | | |
| 09 | N.H. | M | 61 | 15.10.- | HKH, | BurnOut | Iso/PG/P | 2 | 8 | 4 | No | No |
| | | | | 19.11.03 | HYP | | | | | | | |
| 10 | M.L. | F | 48 | 30.11.- | oB | BurnOut | Iso/PG/P | 1 | 8 | 3 | No | No |
| | | | | 15.12.03 | | | | | | | | |
| 11 | G.F. | M | 55 | 10.-31.1.04 | HYP | BurnOut | Iso/PG/P | 2 | 8 | 2 | No | No |
| 12 | S.W. | F | 40 | 25.1.- | HYP | BurnOut | Iso/PG/P | 3 | 7 | 3 | No | No |
| | | | | 25.2.04 | | | | | | | | |
| 13 | B.H. | M | 51 | 1.-27.2.04 | HYP | BurnOut | Iso/PG/P | 2 | 7 | 5 | No | No |
| 14 | F.G. | M | 54 | 11.-31.3.04 | oB | BurnOut | Iso/PG/P | 3 | 8 | 5 | No | No |
| 15 | K.G. | F | 66 | 15.4.-5.5.04 | oB | BurnOut | Iso/PG/P | 2 | 9 | 5 | No | No |
| 16 | M.B. | M | 50 | 20.4.- | HYP | BurnOut | Iso/PG/P | 3 | 7 | 4 | No | No |
| | | | | 15.5.04 | | | | | | | | |
| 17 | S.P. | M | 47 | 3.-31.5.04 | oB | BurnOut | Iso/PG/P | 2 | 8 | 3 | No | No |
| 18 | P.P. | M | 60 | 15.6.- | HYP | BurnOut | Iso/PG/P | 1 | 9 | 4 | No | No |
| | | | | 20.7.04 | | | | | | | | |
| 19 | G.H. | M | 51 | 30.6.- | oB | BurnOut | Iso/PG/P | 1 | 8 | 5 | No | No |
| | | | | 10.8.04 | | | | | | | | |
| 20 | K.B. | M | 56 | 5.7.-5.8.04 | HYP | BurnOut | Iso/PG/P | 2 | 8 | 4 | No | No |

Legend:
oB = without medical finding
HYP = hypertension
KHK = coronary heart disease
DM = diabetes mellitus
Therapy:
Iso = spatial isolation (spa hotel etc.)
PG = psychological interviewing
P = psychotherapy
VAS = visual analogue scale according to Hayes and Patterson (1921), scale 0-10:
0 points: worst impression of the general physical and mental performance
10 points: best individual impression of the general physical and mental performance

From the above Table 3, it can be seen that the patients recovered rapidly and sustainably after receipt of the treatment of the invention. The average VAS value increased from 1.5 before therapy to 10 after therapy. Remarkably, the average VAS value still was 9.8 one year after the therapy.

Furthermore, no additional diseases occurred within 24 months after therapy. The two patients suffering from hypertension additionally showed an improvement of this pre-existing disease.

Further, a control group of patients with diagnosed burnout syndrome was treated by psychotherapy only, i.e. without any medication or similar treatment received. The results based on the VAS evaluation scheme are depicted in table 4. It is noted that the control group, for ethical reasons, had to be treated since burnout syndrome—depending on the severity of the individual case—can be life threatening for the respective patient.

As it can be seen from the data presented in table 4, the VAS after therapy was considerably lower than with the patients treated according to the present invention. Further, the VAS after 12 months tended in many cases to the low value at the start of the therapy thus showing that psychotherapy in many cases was not efficient to treat the burnout syndrome. Finally, not any improvement of preexisting diseases within 24 months could be seen in any of the 20 patients treated in the control group.

Therefore, it has been clearly shown that the therapeutic approach according to the present invention is superior to traditional therapies of the burnout syndrome.

1-12. (canceled)

13. A composition or kit-of-parts comprising or consisting of
a) at least one component comprising a vitamin mixture; and
b) a component comprising human blood plasma IgG antibodies, wherein the vitamin mixture comprises compounds selected from the vitamin groups of vitamin C, vitamin H, vitamin A, vitamin B, vitamin D and vitamin E.

14. The composition or kit-of-parts of claim 13, wherein the vitamin mixture comprises ascorbic acid, biotin, thiamine pyrophosphate, cholecalciferol, cyanocobalamin, panthenol, folic acid, nicotinamide, pyridoxine hydrochloride, retinol palmitate, riboflavin 5′ phosphate, and/or alpha-tocopherol.

15. The composition or kit-of-parts of claim 13, wherein component a) may further comprise minerals such as iron, calcium, magnesium and zinc.

16. The kit-of-parts of claim 13, comprising or consisting of 2 components comprising a vitamin mixture and 1 component comprising IgG antibodies.

17. The composition or kit-of-parts of claim 13, which is in a suitable form for parenteral application, preferably in the form of a solution or suspension for injection or infusion.

18. A method of treatment of burnout syndrome or a method for longevity therapy comprising administering a composition or kit-of-parts comprising or consisting of
a) at least one component comprising a vitamin mixture; and
b) a component comprising human blood plasma IgG antibodies, wherein the vitamin mixture comprises compounds selected from the vitamin groups of vitamin C, vitamin H, vitamin A, vitamin B, vitamin D and vitamin E to a subject in need thereof.

19. The method of treatment of claim 18, wherein the treatment/therapy comprises the following steps:
a) administering a component comprising a vitamin mixture as defined above;
b) administering a component comprising IgG antibodies; and
c) administering a further component comprising said vitamin mixture to a patient in need thereof.

20. The method of treatment of claim 19, where steps a)-c) are performed by intravenous infusion and have a duration of approximately 2 h each.

21. The method of treatment of claim 19, wherein steps a)-c) are repeated 5 times on 5 consecutive days followed by a break of 1-2 days, which in turn is followed by a 5 times repeat of steps a)-c) on 5 consecutive days.

22. The method of treatment of claim 20, wherein steps a)-c) are repeated 5 times on 5 consecutive days followed by a break of 1-2 days, which in turn is followed by a 5 times repeat of steps a)-c) on 5 consecutive days.

23. The method of treatment of claim 18, wherein the patients treated belong to the elderly population.

24. The method of treatment of claim 19, wherein the patients treated belong to the elderly population.
